(19) United States
(12) Patent Application Publication
(10) Pub. No.: US 2006/0183724 A1
(43) Pub. Date: Aug. 17, 2006

(54) Compositions of unconjugated estrogens and methods for their use

(76) Inventors: Charles E. Diliberti, Montclair, NJ (US); Anu Mahashabde, Kendall Park, NJ (US)

Correspondence Address: STERNE, KESSLER, GOLDSTEIN & FOX PLLC, 1100 NEW YORK AVENUE, N.W., WASHINGTON, DC, 20005, US

(21) Appl. No.: 11/346,232

(22) Filed: Feb. 3, 2006

Related U.S. Application Data
(60) Provisional application No. 60/649,077, filed on Feb. 3, 2005.

(51) Int. Cl. A61K 31/56

(52) U.S. Cl. 514/170

(57) ABSTRACT

The present invention relates to compositions containing unconjugated estrogens and methods of their use in the treatment of conditions associated with hypoestrogenism or reduced estrogen levels in females.

This application claims the benefit of the filing date of U.S. Appl. No. 60/649,077, filed Feb. 3, 2005, which is incorporated herein by reference in its entirety.

### BACKGROUND OF THE INVENTION

1. Field of the Invention

The present invention relates to compositions containing unconjugated estrogens and methods of their use in the treatment of conditions associated with hypoestrogenism or reduced estrogen levels in females.

2. Related Art

The primary source of endogenous estrogens in the body of a normally cycling woman is the ovarian follicle. Between the ages of 45 and 55 in women, the ovaries generally stop making estrogens. This drop in body estrogen levels is the cause of the “climacteric” or menopause. If both ovaries are removed during an operation before natural menopause takes place, then the surgical equivalent of menopause, or “surgical menopause,” occurs.

The decrease in estrogen levels that occurs around the time of menopause is primarily responsible for the hot flashes, vaginal atrophy, osteoporosis (Garraway et al., Mayo Clinic Proceedings 54: 701-707 (1979)), and the loss of protection against heart attacks experienced by women (Havlik, R. J. and Manning-Feinleid, P. H., NIH Publication No. 79-1610, U.S. Department of HEW (1979)).

Estrogen replacement therapy (ERT) has been used for symptomatic relief of vasomotor symptoms such as hot flashes or hot flushes, for vaginal atrophy, and for prevention of postmenopausal osteoporosis. The exogenous estrogens provided by ERT modulate the pituitary secretion of the gonadotropins, luteinizing hormone (LH) and follicle stimulating hormone (FSH) through a negative feedback mechanism. Estrogen replacement therapy thus acts to reduce the elevated levels of these hormones seen in postmenopausal women.

Exogenous estrogens are metabolized in the same manner as endogenous estrogens. Circulating estrogens exist in a dynamic equilibrium of metabolic interconversions that occur primarily in the liver. Estradiol is converted reversibly to estrone, and both can be converted to estriol, which is the major urinary metabolite. Estrogens also undergo enterohepatic recirculation via sulfate and glucuronide conjugation in the liver, biliary secretion of conjugates into the intestine, and hydrolysis in the gut followed by reabsorption. In menopausal women, a significant portion of the circulating estrogens exist as sulfate conjugates, especially estrone sulfate, which serve as a circulating reservoir for the formation of more active estrogens.

Conjugated estrogens are used in ERT regimens to treat peri-menopausal and menopausal women who frequently experience a variety of conditions and disorders related to the decrease of estrogen levels in the body. PREMARIN® (conjugated estrogen tablets, USP) is an orally-administered mixture of estrogens obtained exclusively from natural sources, occurring as the sodium salts of water-soluble estrogen sulfates, that is blended to represent the average composition of material derived from pregnant mares' urine. PREMARIN® is indicated for the treatment of moderate to severe vasomotor symptoms associated with menopause, vulvar and vaginal atrophy, and prevention of osteoporosis. A synthetic mix of 9 of the principal estrogenic components of conjugated equine estrogens has also been approved for oral administration in the United States under the tradename CENESTIN®. In both equine-derived conjugated estrogens and in synthetic conjugated estrogens, Type A USP (CENESTIN®), the estrogens are present in water soluble form as the sodium salts of the sulfate esters of the component steroids.

Both PREMARIN® and CENESTIN® contain mixtures of conjugated estrogens. Administering these estrogenic steroids in their water-soluble conjugated forms is beneficial for maximizing their bioavailability when administered orally. This is true because conjugated steroids dissolve readily in gastrointestinal fluids, and the small intestine has large amounts of sulfatase enzymes that cleave the sulfate esters to form the corresponding unconjugated steroid (free steroid) forms, which are readily absorbed by the intestinal mucosa. However, it is the unconjugated (free) forms of steroids that are pharmacologically active species—the conjugated forms are themselves devoid of estrogenic activity, and serve only as prodrugs.

Not all unconjugated estrogens produce identical effects in all target tissues, nor do they necessarily produce precisely the same effects in the same way in different individuals. Thus, a patient might find that a particular unconjugated estrogen or mix of unconjugated estrogens has better efficacy or produces fewer side effects for her than another unconjugated estrogen or another mix of unconjugated estrogens. This is one reason why such a variety of different estrogenic products are commercially produced, such as, for example, preparations of conjugated estrogens, esterified estrogens, piperazine estrone sulfate, 17β-estradiol, or ethinyl estradiol, for oral administration, and preparations of conjugated estrogens, 17β-estradiol, or 17β-estradiol acetate, for vaginal administration.

Thus, a need continues to exist for estrogen preparations having better efficacy and fewer side effects for use in estrogen replacement therapies.

### SUMMARY OF THE INVENTION

The present invention provides compositions containing unconjugated estrogens and methods for their use in the treatment of conditions associated with hypoestrogenism or reduced estrogen levels in females.

Thus, the present invention is directed to an unconjugated estrogen composition comprising two or more unconjugated estrogens, wherein at least one unconjugated estrogen is selected from the group consisting of unconjugated equilin, unconjugated 17α-dihydroequilin, unconjugated 17β-dihydroequilin, and combinations thereof, and wherein the unconjugated estrogen composition is substantially free of conjugated estrogens. In some embodiments, one of the unconjugated estrogens is unconjugated equilin.

In some embodiments, the unconjugated estrogen composition further comprises an unconjugated estrogen selected from the group consisting of unconjugated 17α-estradiol, unconjugated Δ^{8,9}-dehydroestrone, unconjugated 17β-estradiol, unconjugated estrone, unconjugated equilenin, unconjugated 17α-dihydroequilenin, unconjugated 17β-dihydroequilenin and combinations thereof.

In some embodiments, the unconjugated estrogen composition comprises unconjugated equilin and unconjugated estrone.

In some embodiments, the unconjugated estrogen composition is in an anal suppository, buccal, parenteral, transdermal, vaginal, nasal, topical, implantable, or subcutaneous dosage form. In some embodiments, the dosage form has a rate of release of about 0.01 μg/day to about 2000 μg/day of the unconjugated estrogen. In some embodiments, the dosage form has a rate of release of about 0.01 μg/day to about 1000 μg/day of the unconjugated estrogen.

In some embodiments, the unconjugated estrogen composition is in a vaginal dosage form. In some embodiments, the vaginal dosage form is a vaginal ring. In some embodiments, the vaginal ring has a rate of release of about 0.01 μg/day to about 2000 μg/day of the unconjugated estrogen. In some embodiments, the vaginal ring has a rate of release of about 1 μg/day to about 2000 μg/day of the unconjugated estrogen. In some embodiments, the vaginal ring has a rate of release of about 0.01 μg/day to about 1000 μg/day of the unconjugated estrogen. In some embodiments, the vaginal ring has a rate of release of about 1 μg/day to about 1000 μg/day of the unconjugated estrogen. In some embodiments, the vaginal ring has a rate of release of about 1 μg/day to about 500 μg/day of the unconjugated estrogen. In some embodiments, the vaginal ring has a rate of release of about 1 μg/day to about 350 μg/day of the unconjugated estrogen. In some embodiments, the vaginal ring has a rate of release of about 5 μg/day to about 200 μg/day of the unconjugated estrogen. In some embodiments, the vaginal ring comprises a polymer core containing the unconjugated estrogen composition and a polymer sheath that surrounds the polymer core.

The present invention is also directed to a method of treating a peri-menopausal or menopausal condition in a female in need thereof, the method comprising administering the unconjugated estrogen composition in a vaginal ring that releases the unconjugated estrogen at a rate of about 1 μg/day to about 2000 μg/day, about 1 μg/day to about 1000 μg/day, about 1 μg/day to about 500 μg/day, about 1 μg/day to about 350 μg/day, or about 5 μg/day to about 200 μg/day, and wherein the vaginal ring is administered to the female for a period of about 30 days to about 90 days. In some embodiments, the peri-menopausal or menopausal condition is selected from the group consisting of vaginal dryness, vaginal atrophy, vulvar atrophy, atrophic vaginitis, vaginal pruritus, dyspareunia, and dysuria. In some embodiments, the peri-menopausal or menopausal condition is vaginal atrophy.

The present invention is also directed to a method of treating a condition resulting from hypoestrogenism in a female in need thereof, the method comprising administering to the female a therapeutically effective amount of the unconjugated estrogen composition. In some embodiments, the condition resulting from hypoestrogenism is a peri-menopausal or menopausal condition. In some embodiments the peri-menopausal or menopausal condition is selected from the group consisting of vaginal dryness, vaginal atrophy, vulvar atrophy, atrophic vaginitis, vaginal pruritus, dyspareunia, and dysuria. In some embodiments, the peri-menopausal or menopausal condition is vaginal atrophy.

The present invention is also directed to a method of delivering an unconjugated estrogen composition to a patient in need thereof, the method comprising: (a) registering the patient in a computer readable storage medium, and (b) permitting the patient access to the unconjugated estrogen composition. In some embodiments, access to the unconjugated estrogen composition is by a prescription.

### DETAILED DESCRIPTION OF THE INVENTION

The present invention discloses compositions containing unconjugated estrogens and methods for their use in treatment of conditions associated with hypoestrogenism or reduced estrogen levels in females.

Unconjugated Estrogen Compositions

The present invention provides compositions of free, unconjugated estrogens.

A “conjugated estrogen” is an estrogenic steroidal compound in which one or more functional groups (typically hydroxyl groups) on the steroid exist as a conjugate, for example, as a sulfate or glucuronide ester.

The United States Pharmacopeia (USP 23) defines “conjugated estrogens” as a mixture of sodium estrone sulfate and sodium equilin sulfate, derived wholly or in part from equine urine or synthetically from estrone and equilin.

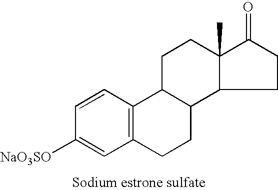

In standardized blends of conjugated estrogens, the most abundant of the conjugated estrogens are the sulfate esters of estrone and equilin. Other conjugated estrogens typically present in such standardized blends are the sodium salts of the sulfate esters of 17-α-dihydroequilin, 17-β-dihydroequilin, and 17-α-estradiol. Also typically present are impurities derived from degradation of the sodium salts of the sulfate esters of the equilin compounds, such as the sodium salts of the sulfate esters of 17-α-dihydroequilenin, 17-β-dihydroequilenin, and equilenin. Other steroids that may be present are the sodium sulfate esters of 17-β-estradiol and Δ^{8,9} dehydroestrone. The USP 23 requires the sodium salts of the sulfate esters of estrone, equilin, 17-α-dihydroequilin, 17-β-dihydroequilin, and 17-α-estradiol to be present in all dosage forms of conjugated estrogens, and these compounds are subject to upper and lower concentration limits in such mixtures.

Conjugated estrogens are known to be chemically unstable and subject to degradation during the manufacturing process. Unconjugated estrogens, in contrast, are more stable and have longer shelf lives than their conjugated analogues.

An “unconjugated estrogen,” as used in the compositions disclosed herein, is an estrogenic steroidal compound in which all functional groups (typically hydroxyl groups) on the steroid are free and unconjugated, e.g., not esterified to form a sulfate ester, glucuronide ester, or other highly polar ester. Unconjugated estrogens can be produced naturally or synthetically. Unconjugated estrogens can also be produced from natural or synthetic sources.

Estrone is an example of an unconjugated estrogen. This unconjugated estrogen lacks the sulfate moiety present on the C-3 carbon of the steroid ring that is present in the conjugated form of estrone, sodium estrone sulfate.

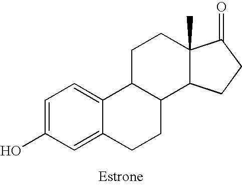

Other examples of unconjugated estrogens include, but are not limited to, the free, unconjugated forms of what the USP defines as conjugated estrogens, such as unconjugated equilin, unconjugated 17α-dihydroequilin, unconjugated 17β-dihydroequilin, unconjugated 17α-estradiol, unconjugated Δ^{8,9}-dehydroestrone, unconjugated 17β-estradiol, unconjugated equilenin, unconjugated 17α-dihydroequilenin, and unconjugated 17β-dihydroequilenin; estradiol valerate; estradiol benzoate; estradiol 17β-cyprionate; estradiol 17-propionate; estradiol acetate; and mestranol.

As used herein, the term “unconjugated estrogen” refers to a single unconjugated estrogen compound or to a mixture of two or more unconjugated estrogen compounds. The term “unconjugated estrogen composition” refers to a composition containing one unconjugated estrogen compound or to a composition containing a mixture of two or more unconjugated estrogen compounds.

The unconjugated estrogen compositions of the present invention can contain one unconjugated estrogen or a mixture of a variety of natural and/or synthetic unconjugated estrogens. However, the unconjugated estrogen compositions contain at least one or more unconjugated estrogens selected from unconjugated equilin, unconjugated 17α-dihydroequilin, and unconjugated 17β-dihydroequilin. Additional unconjugated estrogens that can be present in the unconjugated estrogen compositions include, but are not limited to, one or more of the following: unconjugated 17α-estradiol, unconjugated Δ^{8,9}-dehydroestrone, unconjugated 17β-estradiol, unconjugated estrone, unconjugated equilenin, unconjugated 17α-dihydroequilenin, and unconjugated 17β-dihydroequilenin. The unconjugated estrogen compositions also can contain a pharmaceutically acceptable carrier.

For example, in some aspects of the invention, the unconjugated estrogen compositions comprise equilin and estrone as the only estrogens. In other aspects, the unconjugated estrogen compositions comprise unconjugated equilin, unconjugated 17α-dihydroequilin, unconjugated 17β-dihydroequilin, unconjugated 17α-estradiol, unconjugated equilenin, unconjugated 17α-dihydroequilenin, unconjugated 17β-dihydroequilenin, unconjugated 17β-estradiol and unconjugated estrone. In yet other aspects, the unconjugated estrogen compositions comprise equilin, estrone, and additional unconjugated estrogens, with equilin and estrone as the predominant unconjugated estrogens.

The unconjugated estrogen compositions of the present invention are substantially free of conjugated estrogens. Thus, the unconjugated estrogen compositions of the invention are free of any physiologically significant amounts of conjugated estrogens. The term “substantially free” as used herein refers to the presence of less than 10% by weight of conjugated estrogens, less than 5% by weight of conjugated estrogens, less than 1% by weight of conjugated estrogens, less than 0.50% by weight of conjugated estrogens, less than 0.25% by weight of conjugated estrogens, or less than 0.1% by weight of conjugated estrogens.

Thus, the present invention is directed to an unconjugated estrogen composition comprising an unconjugated estrogen selected from the group consisting of unconjugated equilin, unconjugated 17α-dihydroequilin, unconjugated 17β-dihydroequilin, and combinations thereof; wherein the unconjugated estrogen composition is substantially free of conjugated estrogens. In some aspects of the invention, the unconjugated estrogen composition further comprises one or more unconjugated estrogens selected from the group consisting of unconjugated 17α-estradiol, unconjugated Δ^{8,9}-dehydroestrone, unconjugated 17β-estradiol, unconjugated estrone, unconjugated equilenin, unconjugated 17α-dihydroequilenin, unconjugated 17β-dihydroequilenin, and combinations thereof.

In some aspects of the invention, the unconjugated estrogen composition further comprises a pharmaceutically acceptable carrier. Thus, the present invention is also directed to an unconjugated estrogen composition comprising an unconjugated estrogen selected from the group consisting of unconjugated equilin, unconjugated 17α-dihydroequilin, unconjugated 17β-dihydroequilin, and combinations thereof; and a pharmaceutically acceptable carrier; wherein the unconjugated estrogen composition is substantially free of conjugated estrogens.

The present invention is also directed to an unconjugated estrogen composition comprising a first unconjugated estrogen selected from the group consisting of unconjugated equilin, unconjugated 17α-dihydroequilin, unconjugated 17β-dihydroequilin, and combinations thereof; and a second unconjugated estrogen selected from the group consisting of unconjugated 17α-estradiol, unconjugated Δ^{8,9}-dehydroestrone, unconjugated 17β-estradiol, unconjugated estrone, unconjugated equilenin, unconjugated 17α-dihydroequilenin, unconjugated 17β-dihydroequilenin, and combinations thereof; wherein the unconjugated estrogen composition is substantially free of conjugated estrogens. In some aspects of the invention, the unconjugated estrogen composition comprising the first unconjugated estrogen and the second unconjugated estrogen further comprises a pharmaceutically acceptable carrier.

Table I provides exemplary weight percent ranges for each unconjugated estrogen in compositions of the invention that contain equilin, estrone, and one or more unconjugated estrogens selected from 17α-estradiol, 17α-dihydroequilin, 17β-dihydroequilin, 17β-estradiol, 17α-dihydroequilenin, 17β-dihydroequilenin, equilenin, and Δ^{8,9} dehydroestrone.

TABLE I
Unconjugated Estrogen Compositions

| | Weight Percent (% w/w) of the |
|---|---|
| | Total Amount of Unconjugated |
| | Estrogens in the Composition |
| Unconjugated Estrogens | |
| Estrone | 52.5-61.5 |
| Equilin | 22.5-30.5 |
| Sum of Equilin and Estrone | 79.5-88.0 |
| 17α-Estradiol | 2.5-9.5 |
| 17α-Dihydroequilin | 13.5-19.5 |
| 17β-Dihydroequilin | 0.5-4.0 |
| Unconjugated Estrogen Impurities | |
| Or Other Substances | |
| 17β-Estradiol | ≦2.25 |
| 17α-Dihydroequilenin | ≦3.25 |
| 17β-Dihydroequilenin | ≦2.75 |
| Equilenin | ≦5.50 |
| Δ^{8,9} Dehydroestrone | ≦6.25 |

The individual unconjugated estrogens in the compositions of the present invention are known to those of skill in the art and are available commercially (e.g., from Sigma-Aldrich, St. Louis, Mo.; ORGANICS/LaGrange, Inc., Northbrook, Ill.; or Diosynth, Inc., Des Plaines, Ill.) or can be prepared by any one of various known methods (see, e.g., “The Total Synthesis of Natural Products,” Vol. 2, pp. 642-663, ApSimon, Ed. (John Wiley & Sons, New York, 1973). Such methods also include chemical or enzymatic hydrolysis of the naturally occurring mixtures of conjugated estrogens derived from the purified urine of pregnant mares.

When administered according to the invention, the unconjugated estrogen compositions can be administered, e.g., continuously and uninterruptedly during the treatment period. In some aspects of the invention, the unconjugated estrogen compositions are administered continuously and uninterruptedly for about 30 days. In other aspects, the unconjugated estrogen compositions are administered continuously and uninterruptedly for about 30 days to about 90 days. In yet other aspects of the invention, the unconjugated estrogen compositions are administered continuously and uninterruptedly for about 120 days, or for at least one year, or for more than one year.

The term “continuous,” “consecutive,” or “continuously and uninterruptedly” in reference to administration means that there is no break in the treatment regimen during the treatment period. Thus, “continuous,” “consecutive,” or “continuous and uninterrupted” administration means that the frequency of administration is at least once daily. Note, however, that the frequency of administration can be greater than once daily and still be “continuous,” e.g., twice or even three times daily, as long as the dosage levels as specified herein are not exceeded. Moreover, if a patient misses or forgets to take one or a few dosages during the course of a treatment regimen, such patient is still considered to be receiving “continuous,” or “continuous and uninterrupted,” administration.

The treatment period can vary depending on a number of factors, including, but not limited to, the condition or disorder to be treated, the physiological effect desired, and the mode of administration of the unconjugated estrogen compositions, and can be determined readily by one of skill in the medical arts.

Thus, for example, when administered for the treatment of vasomotor symptoms, e.g., vaginal atrophy, the unconjugated estrogen compositions of the invention can be administered continuously and uninterruptedly for a period of from about one month to about several years, depending on the severity and duration of the symptoms. When administered for the treatment or inhibition of osteoporosis, the unconjugated estrogen compositions can be administered continuously and uninterruptedly for a period of about six months to a number of years.

As with the treatment period, the actual dosage administered will be dependent upon a number of factors, including, but not limited to, the condition or disorder to be treated, the physiological effect desired, and the mode of administration of the disclosed unconjugated estrogen compositions, and can be determined readily by one of skill in the medical arts.

For example, in some aspects of the invention, the unconjugated estrogen compositions are administered in dosages of about 0.0001 mg (0.1 μg) of unconjugated estrogens per day to about 10.0 mg unconjugated estrogens per day. In other aspects, the unconjugated estrogen compositions are administered in dosages of about 0.002 mg (2 μg) mg of unconjugated estrogens per day to about 2.0 mg unconjugated estrogens per day, or about 0.005 mg of unconjugated estrogens per day to about 0.200 mg unconjugated estrogens per day. In yet other aspects, the unconjugated estrogen compositions are administered in dosages of about 0.005 mg of unconjugated estrogens per day, in dosages of about 0.050 mg of unconjugated estrogens per day, or in dosages of about 0.100 mg of unconjugated estrogens per day. In other aspects of the invention, the unconjugated estrogen compositions are administered in dosages of about 0.150 mg of unconjugated estrogens per day, or in dosages of about 0.200 mg of unconjugated estrogens per day.

The term “dose,” “dosage level,” “dosage,” or “daily dosage” means the total amount of unconjugated estrogens administered per day. Thus, for example, “continuous administration,” or “continuous and uninterrupted administration,” of unconjugated estrogens to a woman at a “dosage level” or “dosage” of 0.030 mg means that the woman receives a total of 0.030 mg of unconjugated estrogens on a daily basis, whether the unconjugated estrogens are administered as a single 0.030 mg dose, as multiple doses per day (e.g., as three separate 0.010 mg doses), or as a single dosage form or multiple dosage forms with a total rate of release of 0.030 mg per day.

Methods of Treatment

The unconjugated estrogen compositions of the present invention are useful for treatment of conditions associated with hypoestrogenism or reduced estrogen levels in females. Thus, for example, the unconjugated estrogen compositions can be administered to peri-menopausal women and/or menopausal women to treat any of a number of conditions that often result from decreases in the production of endogenous estrogen that occur as a woman nears or achieves menopause, such as vaginal atrophy.

One advantage of the present invention is that the unconjugated estrogen compositions may offer superior efficacy or fewer side effects for certain individual patients than other commercially available preparations. For those individual patients who prefer to use an estrogen mix similar to that in conjugated estrogens vaginal cream, the unconjugated estrogen compositions of the present invention may offer them the enhanced benefit of this estrogen mix in a number of formulations, including a vaginal ring formulation.

As used herein, “female” refers to any animal classified as a mammal, including humans and non-humans, such as, but not limited to, domestic and farm animals.

The term “effective amount” or “therapeutically effective amount,” as used herein, refers to the amount of the unconjugated estrogen composition that is effective to achieve its intended purpose after a single dose or after a course of doses, e.g., during or at the end of the treatment period. Thus, for example, the term “therapeutically effective amount” of the unconjugated estrogen composition, when used in a method of treating a peri-menopausal symptom such as hot flashes, refers to that dose of the unconjugated estrogen composition that lessens or prevents the occurrence of hot flashes when administered to the female in need of such treatment.

“Peri-menopausal female” refers to a woman who has not yet definitely arrived at menopause but who is experiencing symptoms associated with menopause. “Peri-menopause” means “about or around the time of menopause.” It encompasses the years preceding the last menstrual period during which ovarian function declines and ultimately ceases and can include the presence of symptoms and irregular cycles. “Menopausal female” refers to a woman who has definitely arrived at menopause and may be experiencing symptoms associated with menopause. Menopause is the permanent cessation of menstruation after the loss of ovarian activity and is generally defined clinically as the absence of menstruation for about one year. Menopause can occur naturally in a woman or it can be artificially induced, e.g., through surgical or chemical means. For example, removal of the ovaries, which can occur through hysterectomy, frequently leads to symptoms associated with menopause (“surgical menopause”).

The term “peri-menopausal condition” refers to a condition, disorder, or disease state, that is at least partially caused by the decreased estrogen production in a peri-menopausal female that occurs prior to menopause. The term “peri-menopausal symptom” refers to a symptom associated with a peri-menopausal condition. The term “menopausal condition” refers to a condition, disorder, or disease state that is at least partially caused by the decreased estrogen production in a menopausal or post-menopausal woman that occurs during menopause and in the years following menopause. The term “menopausal symptom” refers to a symptom associated with a menopausal condition. Peri-menopausal and menopausal conditions and disorders include, but are not limited to, vasomotor symptoms, such as hot flashes and flushes, myalgia, arthralgia, depression, insomnia, and nervousness; vaginal dryness; vaginal or vulvar atrophy; atrophic vaginitis; vaginal pruritus; dyspareunia; dysuria; frequent urination; urinary incontinence; urinary tract infections; dementias; neurodegenerative disorders such as Alzheimers' disease; osteopenia; and osteoporosis.

The terms “treat,” “treatment,” and the like, as used herein, refer to therapeutic treatment, wherein the object is to reduce, slow or reverse an undesired physiological condition, disorder or disease, or obtain beneficial or desired clinical results. These terms also refer to prophylactic or preventative measures, wherein the object is to prevent an undesired physiological condition, disorder or disease from occurring. For purposes of this invention, beneficial or desired clinical results include, but are not limited to, alleviation of symptoms; diminishment of extent of the condition, disorder or disease; stabilization (i.e., no further worsening) of the state of the condition, disorder or disease; delay in onset, or slowing, of the condition, disorder or disease progression; amelioration of the condition, disorder or disease state; and remission (whether partial or total) or improvement of the condition, disorder or disease. Treatment includes eliciting a clinically significant response, preferably without unacceptable levels of side effects. Treatment also includes prolonging survival as compared to expected survival if not receiving treatment.

Peri-menopausal and menopausal women frequently experience a variety of conditions and disorders that have been attributed to estrogen deprivation due to ovarian failure or hypoestrogenism. The duration of these disorders can be extremely variable and include vasomotor symptoms that include, but are not limited to, hot flushes, myalgia, arthralgia, depression, insomnia, and nervousness, which can be devastating in some women and very mild in others. Dryness of the vagina and increased vaginal pH associated with susceptibility to minor infections, and frequently associated with discomfort during intercourse, is another symptom that can be directly related to the decrease in estrogen availability. Other conditions or disorders include, but are not limited to, vaginal or vulvar atrophy, atrophic vaginitis, vaginal pruritus, dyspareunia, dysuria, frequent urination, urinary incontinence, and urinary tract infections.

One of the most health-threatening long-term aspects of menopause is the loss of mineral from bone which can result in a decrease in bone mass (osteoporosis) and generates a serious risk of fractures. For example, evidence exists that there is a six-fold increase in fractures in post-menopausal women as opposed to men of the same age (Garraway et al., Mayo Clinic Proceedings 54:701-707 (1979)). These fractures carry a high complication rate among older people, a marked increase in disability and general morbidity, and certainly an increased risk of mortality.

Another serious health-threatening aspect of menopause is the impressive loss of protection against heart attacks, which is enjoyed by younger women up to the age of 60, when compared to men of the same age. The steep increase in mean serum cholesterol concentration, which occurs around menopause (during the fourth and fifth decades), can contribute importantly to the progressive increase in death from ischemic heart disease in older women. In the eighth and ninth decades, the incidence of deaths from ischemic heart disease, approaches that of men (Havlik, R. J. and Manning-Feinleid, P. H., NIH Publication No. 79-1610, U.S. Department of HEW (1979)).

Estrogen replacement therapy (ERT) has been used for symptomatic relief of vasomotor symptoms such as hot flashes and hot flushes, and genital atrophy and for prevention of postmenopausal osteoporosis. There has been no acceptable alternative to estrogen treatment for the atrophic changes in the vagina; estrogen therapy decreases vaginal pH, increases the proportion of superficial cells in the vaginal epithelium, and decreases vaginal dryness. Long term ERT is key to preventing osteoporosis because it decreases bone loss, reduces spine and hip fracture, and prevents loss of height. In addition ERT has been shown to be effective in increasing high density lipoprotein-cholesterol (HDL-C), affording possible protection against cardiovascular disease. ERT also can provide antioxidant protection against free radical mediated disorders or disease states. Estrogens have also been reported to confer neuroprotection, and inhibit neurodegenerative disorders, such as Alzheimer's disease (see U.S. Pat. No. 5,554,601, which is hereby incorporated by reference).

Accordingly, the present invention is directed to a method for treating conditions such as, for example, the conditions described above, resulting from menopausal estrogen decline, hypoestrogenism, or ovarian failure in a female by administering to the female a therapeutically effective amount of an unconjugated estrogen composition of the present invention.

Thus, the invention is directed to a method of treating a menopausal or peri-menopausal condition in a female in need thereof by administering to the female a therapeutically effective amount of an unconjugated estrogen composition of the present invention. The unconjugated estrogen compositions can be administered to the female to treat menopausal or peri-menopausal conditions including: vasomotor symptoms, such as hot flushes, myalgia, arthralgia, depression, insomnia, and nervousness; vaginal dryness; vaginal or vulvar atrophy; atrophic vaginitis; vaginal pruritus; dyspareunia; dysuria; frequent urination; urinary incontinence; and urinary tract infections.

The invention is also directed to a method of treating a condition resulting from menopausal or peri-menopausal estrogen decline, including osteoporosis, in a female in need thereof by administering to the female a therapeutically effective amount of an unconjugated estrogen composition of the present invention. Thus, the unconjugated estrogen compositions can be used in a method of treating osteoporosis, or in a method of maintaining bone density or preventing loss of bone density, in a female. The unconjugated estrogen compositions can also be used in this way by administering an effective amount of, e.g., calcium and/or vitamin D in combination with the administration of the unconjugated estrogen compositions. Thus, the invention is directed to a method of maintaining bone density or preventing loss of bone density in a female by administering to the female a therapeutically effective amount of an unconjugated estrogen composition disclosed herein.

Administration of the unconjugated estrogen compositions to maintain bone density or prevent bone loss is not limited to peri-menopausal or menopausal females. The unconjugated estrogen compositions can also be used in a method of maintaining bone density or preventing bone loss by administration to a female of childbearing age that is not peri-menopausal or menopausal. For example, the unconjugated estrogen compositions can be used with females 12-16 years of age who have not yet achieved peak bone density, but who, due to various conditions such as anorexia, are at risk of loss of bone density or at risk of not achieving a normal physiologic bone density for age and developmental maturity.

The unconjugated estrogen compositions can also be used in a method of treating a female in need of estrogen replacement therapy. Thus, the invention is also directed to a method of treating a female in need of estrogen replacement therapy by administering a therapeutically effective amount of an unconjugated estrogen composition of the present invention.

The unconjugated estrogen compositions of the invention, when combined with a progestin, also exert a cardioprotective effect in peri-menopausal and menopausal women and are therefore useful in lowering cholesterol, lipoprotein A, and LDL (low density lipoprotein) levels; increasing HDL (high density lipoprotein) levels; inhibiting or treating hypercholesteremia, hyperipidemia, cardiovascular disease, atherosclerosis, peripheral vascular disease, restenosis, and/or vasospasm; and in inhibiting vascular wall damage from cellular events leading toward immune mediated vascular damage.

Thus, the invention is also directed to a method of treating hypercholesteremia, hyperipidemia, cardiovascular disease, atherosclerosis, peripheral vascular disease, restenosis, and/or vasospasm in a female in need thereof by administering a therapeutically effective amount of an unconjugated estrogen composition of the present invention. The invention is also directed to a method of lowering cholesterol, lipoprotein A, and LDL (low density lipoprotein) levels, or of increasing HDL (high density lipoprotein) levels, in a female in need thereof by administering a therapeutically effective amount of an unconjugated estrogen composition of the present invention.

The unconjugated estrogen compositions of the invention also can be used in a method of treating dementias, and/or neurodegenerative disorders such as Alzheimers' disease, and in providing neuroprotection or cognition enhancement, in peri-menopausal and menopausal females. Thus, the invention is also directed to a method of treating dementias and/or neurodegenerative disorders in a female in need thereof by administering a therapeutically effective amount of an unconjugated estrogen composition of the present invention.

The unconjugated estrogen compositions of the invention can also be used in a method of treating alopecia in peri-menopausal and menopausal females in need thereof. Thus, the present invention is also directed to a method of treating alopecia in a female in need thereof by administering a therapeutically effective amount of an unconjugated estrogen composition of the present invention.

Modes of Administration, Formulations, and Kits

The unconjugated estrogen compositions of the present invention can be combined with a pharmaceutically acceptable carrier to make an unconjugated estrogen pharmaceutical composition. The unconjugated estrogens in such pharmaceutical compositions can comprise from about 0.001% to about 99% (w/w) of the pharmaceutical composition. Pharmaceutically acceptable carriers include, but not limited to, pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like, as well as suitable solid or gel phase carriers or excipients. Examples of such carriers or excipients include, but are not limited to, calcium carbonate, calcium phosphate, various sugars, starches, cellulose derivatives, gelatin, and polymers such as, e.g., polyethylene glycols, or polymers such as polyolefins, polyvinylchoride, polyurethanes, polyamides, or elastomers, e.g., silicone polymers, that are used in the formation of intrauterine devices and vaginal rings.

The unconjugated estrogen compositions can be administered by intravaginal, transdermal, parenteral, subcutaneous, intravenous, intramuscular, intranasal, buccal, rectal, or ocular routes, or by inhalation, by depot injections, or by hormone implants. Thus, modes of administration for the unconjugated estrogen compositions of the present invention (either alone or in combination with other active substances) can be, but are not limited to, use of vaginal creams, suppositories, pessaries, vaginal rings, rectal suppositories, intrauterine devices, and transdermal forms such as patches and creams; or by injection (including short-acting, depot, implant and pellet forms injected subcutaneously or intramuscularly).

The means and methods of any of these modes of administration are known in the art and an artisan can refer to various pharmacologic references for guidance. For example, “Modern Pharmaceutics”, Banker & Rhodes, Marcel Dekker, Inc. 1979; and “Goodman & Gilman's The Pharmaceutical Basis of Therapeutics,” 6^{th} Edition, MacMillan Publishing Co., New York 1980 can be consulted. Pharmaceutical formulations containing the disclosed unconjugated estrogen compositions can be solid dosage forms which include, but are not limited to, tablets, capsules, cachets, pellets, powders, granules, and suppositories; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; comprising an effective amount of the unconjugated estrogen compositions of the invention.

The term “pharmaceutically acceptable,” as used herein in reference to a carrier, diluent, excipient, and the like means that the carrier, diluent or excipient is compatible with the other ingredients of the composition or formulation and not deleterious to the recipient.

In some aspects of the invention, the unconjugated estrogen compositions are administered transdermally. For the purposes of this disclosure, “transdermal administration” is understood to include any administration across the surface of the body, including the outer epithelial surface as well as the inner linings of bodily passages, including mucosal tissues. Such administrations may be carried out using the disclosed unconjugated estrogen compositions of the invention in, for example, lotions, creams, foams, patches, suspensions, solutions, and suppositories (rectal and vaginal).

In some aspects of the invention, the unconjugated estrogen compositions are administered vaginally. When administered vaginally, the disclosed unconjugated estrogen compositions can take any of a variety of forms that include, but are not limited to, vaginal creams, suppositories, gels, tablets, pessaries, vaginal rings, and intrauterine devices.

Vaginally administered unconjugated estrogen compositions of the present invention possess an advantage over vaginally administered compositions of conjugated estrogens. When conjugated estrogens are administered vaginally, sulfatase enzymes present in the vaginal wall are necessary to convert the conjugated estrogens to their unconjugated, pharmacologically active forms. The amount of sulfatase enzymes present in the vaginal wall, however, may not be sufficient to reliably convert the entire dose of conjugated estrogens to their active forms. Thus, the fraction of the dose of conjugated estrogens that is actually available to the target tissue (vaginal epithelium) varies significantly from one individual to the next. Direct vaginal absorption of conjugated estrogens is also limited due to the hydrophilic nature of the charged sulfate group present in the conjugated forms.

Unconjugated estrogens, in contrast, are hydrophobic and thus much more readily absorbed across the vaginal mucosa than conjugated estrogens. Administering estrogens vaginally in their unconjugated forms, as disclosed herein, rather than in their conjugated forms also circumvents the problem of relying upon limited endogenous sulfatase activity to effect delivery of pharmacologically active estrogens to the vaginal epithelium. Thus, the use of vaginally administered unconjugated estrogen compositions of the invention reduces the variability of drug delivery to the vaginal epithelium that occurs with vaginally administered conjugated estrogen preparations.

Methods of making vaginal creams, gels, and suppositories for administering the disclosed unconjugated estrogen compositions are known in the art. For example, vaginal creams can be formulated to contain the disclosed unconjugated estrogen compositions in a nonliquefying base containing cetyl ester wax, cetyl alcohol, white wax, glyceryl monostearate, propylene glycol monostearate, methyl stearate, benzyl alcohol, sodium lauryl sulfate, glycerin, and/or mineral oil as excipients.

Methods for making vaginal rings and intrauterine devices are known or discussed in the art. See, for example, U.S. Pat. No. 6,554,546 (Groenewegen et al.), U.S. Pat. No. 6,394,094 (McKenna et al.), U.S. Pat. No. 6,126,958 (Saleh et al.), U.S. Pat. No. 6,039,968 (Nabahi), U.S. Pat. No. 5,989,581 (Groenewegen), U.S. Pat. No. 5,788,980 (Nabahi), U.S. Pat. No. 5,694,947 (Lehtinen et al.), U.S. Pat. No. 5,188,835 (Lindskog et al.), U.S. Pat. No. 4,822,616 (Zimmermann et al.), U.S. Pat. No. 4,596,576 (de Nijs), and U.S. Appl. Pub. No. US 2003/0060785 (Lavean et al.). See also, for example, U.S. Pat. No. 5,972,372 (Saleh et al.), U.S. Pat. Nos. 4,012,496 and 4,155,991 (Schopflin et al.), U.S. Pat. No. 5,855,906 (McClay), and U.S. Pat. Nos. 4,629,449 and 4,402,695 (Wong). Each of these documents is fully incorporated herein by reference in its entirety.

Vaginal rings incorporating the unconjugated estrogen compositions of the present invention can be made in any of the designs known to those of skill in the art. For example, in the ring design, the active ingredient can be distributed throughout a hydrophobic elastomeric matrix. In the core design, the active ingredient can be dispersed in a polymer matrix core surrounded by a rate-controlling sheath. In the shell design, the active ingredient can be contained in a narrow band between a non-medicated central hydrophobic elastomeric core and an outer sheath. The outer sheath acts as a rate-controlling membrane. Vaginal rings are typically constructed of biocompatible polymers or mixtures of polymers, such as, for example, polyolefins (e.g., polyethylene and polypropylene), polyurethanes, ethylene/vinylacetate copolymers, and silicone elastomers such as organopolysiloxanes. See, for example, U.S. Pat. Nos. 6,126,958 and 5,989,581.

The unconjugated estrogen compositions can be incorporated into vaginal rings by any of the methods known in the art. For example, the disclosed unconjugated estrogen compositions can be incorporated into vaginal rings by the co-injection methods described in McKenna et al., U.S. Pat. No. 6,394,094, the contents of which are fully incorporated herein by reference.

Vaginal rings containing the unconjugated estrogen compositions can typically release the incorporated unconjugated estrogens continuously at a rate of about 5 μg/day to about 200 μg per day. When administered to a female according to the present invention, each vaginal ring is typically used by the female for about 30 to about 90 days, after which it is replaced with a new vaginal ring if treatment is to be continued.

Use of vaginal rings results in high patient compliance and patient convenience because each ring is administered to the patient only once every 30-90 days. Vaginal rings are likely to stay in place within the patient and result in no loss of dose. Because the release of unconjugated estrogens is continuous over each 30-90 day period, use of a vaginal ring minimizes fluctuations in serum levels of unconjugated estrogens.

In some aspects of the invention, the unconjugated estrogen compositions of the present invention are formulated as a transdermal patch.

The preparation and use of transdermal patches are known or discussed in the art. See, for example, U.S. Pat. Nos. 6,214,374 (Schmirler et al.), 6,156,335 (Rovati et al.), 6,153,216 (Cordes et al.), 6,071,531 (Jona et al.), 5,762,956 and 5,145,682 (Chien et al.), 5,474,783 (Miranda et al.), 5,006,342 (Cleary et al.), and U.S. Appl. Pub. No. US 2003/0175328 (Shefer et al.). See also U.S. Pat. No. 4,460,372 (Campbell et al.), U.S. Pat. No. 4,573,996 (Kwiatek et al.), U.S. Pat. No. 4,624,665 (Nuwayser), U.S. Pat. No. 4,722,941 (Eckert et al.), and U.S. Pat. No. 5,223,261 (Nelson et al.). Each of these documents is hereby incorporated by reference in its entirety.

Transdermal patches are available in different designs, any of which can be used to administer the unconjugated estrogen compositions of the invention. Most transdermal patches fall into one of two categories based on design: reservoir-type or matrix-type. A reservoir-type patch typically comprises a reservoir containing the active agent to be administered, a non-permeable backing layer covering one side of a reservoir, and a permeable surface layer and adhesive layer covering the other side of the reservoir. The adhesive layer covers the surface of the permeable surface layer on the side opposite the reservoir. The reservoir contains the active agent in solution or in a gel that is in direct contact with the permeable surface layer, which acts as a rate-controlling membrane that controls delivery of the active agent to the skin of the subject. The adhesive layer adheres the transdermal patch to the subject's skin. When the transdermal patch is placed in contact with the subject's skin, the active agent in the reservoir passes through the permeable surface layer and the adhesive layer and penetrates the skin, passing into the systemic circulation. Reservoir-type transdermal patches can include one or more penetration-enhancing agents in the reservoir that enhance the penetration of the active agent through the skin. See, for example, U.S. Pat. No. 6,214,374 (Schmirler et al.), the contents of which is fully incorporated by reference herein.

Examples of suitable materials that can be used to manufacture reservoir-type transdermal patches are known in the art of transdermal patch delivery. Examples of suitable materials that can form the backing layer of a reservoir-type patch are known to those of skill in the art, and any conventional backing layer material can be employed. Examples of suitable permeable surface layer materials are also known (see, for example, U.S. Pat. No. 3,797,494 (Zaffaroni)). Suitable adhesives that can be coated onto the backing layer to form the adhesive layer are also known. Suitable penetration-enhancing agents are known in the art as well. For examples of specific penetration-enhancing agents, see Williams, A., and Barry, B., Adv. Drug Delivery Rev. 56:603-618 (2004), which is incorporated by reference herein in its entirety.

Examples of matrix-type patches are also well known to those of skill in the art and include, for example, the CLIMARA® patch available from Berlex, and the ORTHO-EVRA® patch available from Ortho-McNeil. In general, a matrix-type transdermal patch comprises a backing layer and an adhesive/drug layer. The adhesive/drug layer has the combined function of adhering the patch to the skin of the subject and holding the active agent to be administered. When the patch is placed in contact with the subject's skin, the active agent is leached from the adhesive/drug layer to and through the skin, passing into the systemic circulation. For examples of a matrix-type transdermal patch, see U.S. Pat. No. 5,972,377, which is incorporated by reference herein in its entirety.

The transdermal patches employed with the unconjugated estrogen compositions can also include a variety of additional excipients that are conventionally employed to facilitate the transdermal administration of an active agent, particularly a steroidal active agent. Examples of such excipients include, but are not limited to, carriers, gelling agents, suspending agents, dispersing agents, preservatives, stabilizers, wetting agents, emulsifying agents, and the like. Specific examples of each of these types of excipients are well known in the art of transdermal drug delivery and any conventional excipients can be employed.

In other aspects of the invention, the unconjugated estrogen compositions are formulated for buccal administration. For buccal administration, the disclosed unconjugated estrogen compositions can take the form of, e.g., tablets or lozenges formulated in a conventional manner. In other aspects of the invention, the disclosed unconjugated estrogen compositions are formulated in rectal compositions such as suppositories or retention enemas, e.g., containing conventional suppository bases such as cocoa butter or other glycerides.

In yet other aspects of the invention, the unconjugated estrogen compositions are formulated for administration by inhalation. When formulated for administration by inhalation, the disclosed unconjugated estrogen compositions are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin, for use in an inhaler or insufflator can be formulated containing a powder mix of the disclosed unconjugated estrogen compositions and a suitable powder base such as lactose.

In some aspects of the invention, the unconjugated estrogen compositions are formulated as an injectable depot preparation. Such long acting formulations can be administered by implantation (for example subcutaneously or intramuscularly) or by intramuscular injection. Depot injections can be administered at about 1 to about 6 months or longer intervals. Thus, for example, the disclosed unconjugated estrogen compositions can be formulated with suitable polymeric or hydrophobic materials (for example as an emulsion in an acceptable oil) or ion exchange resins, or as sparingly soluble derivatives, for example, as a sparingly soluble salt.

In other aspects of the invention, the unconjugated estrogen compositions are formulated for parenteral administration by injection, e.g., by bolus injection or continuous infusion. The disclosed unconjugated estrogen compositions can be administered by continuous infusion subcutaneously over a period of about 15 minutes to about 24 hours. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

The unconjugated estrogen compositions of the present invention can be administered with other pharmaceutically active agents or compounds. For example, in some aspects of the invention, the unconjugated estrogen compositions are optionally administered with one or more progestins. Examples of suitable progestins include, but are not limited to, progesterone and progesterone derivatives such as medroxyprogesterone, medroxyprogesterone acetate, and megestrol acetate, as well as chlormadinone acetate, norethindrone, cyproterone acetate, norethindrone acetate, desogestrel, levonorgestrel, drospirenone, trimegestone, norgestrel, norgestimate, norelgestromin, etonogestrel, gestodene, and other natural and/or synthetic gestagens.

Examples of other additional pharmaceutically active agents that can be optionally administered with the unconjugated estrogen compositions include, but are not limited to, vitamin D or vitamin D analogues; one or more of the B complex vitamins, such as vitamin B3 (niacin, nicotinic acid and/or nicotinamide), vitamin B9 (folic acid or folate), vitamin B6 and/or vitamin B12; minerals such as, for example, calcium; bisphosphonates (e.g., alendronate); teriparatide (e.g., FORTEO™); and SERMs (selective estrogen receptor modulators, e.g., raloxifene). For example, the unconjugated estrogen compositions can be administered with vitamin D and/or calcium or a bisphosphonate as a method of maintaining or preventing loss of bone density. Suitable forms of vitamin D and of calcium and bisphosphonate would be known to those of skill in the art.

The unconjugated estrogen compositions of the invention can also be optionally administered with an antidepressant, such as, for example, a selective serotonin reuptake inhibitor (SSRI), a tricyclic antidepressant or anxiolytic, or any antidepressant known to one of skill in the art. Suitable antidepressants include, but are not limited to, alprazolam (XANAX®), clomipramine (ANAFRANIL®), fluoxetine (PROZAC®), paroxetine (PAXIL®), sertraline (ZOLOFT®), nefazodone (SERZONE®), and venlafaxine (EFFEXOR®).

The additional pharmaceutically active agent administered with the unconjugated estrogen compositions can be administered using any suitable modes of administration, including, but not limited to, parenteral, oral, buccal, rectal, subcutaneous, intravenous, intramuscular, intranasal, transdermal modes of administration, and by inhalation. In some aspects of the invention, the additional active agent is administered using the same mode of administration as the unconjugated estrogen composition. For example, the additional active agent, e.g., a progestin, and the unconjugated estrogen composition are administered together using the same mode of administration, either in the same dosage form (e.g., transdermally, using the same vaginal ring) or, alternatively, in two different dosage forms (e.g., as two separate vaginal creams). In other aspects, the additional active agent is administered using a different mode of administration, e.g., the unconjugated estrogen composition is administered transdermally, using a transdermal delivery device such as a vaginal ring, and the additional active agent, e.g., an antidepressant, is administered orally, in the form of a pill or tablet.

The dosage of the additional active agent or compound can be determined readily by one of skill in the medical arts and will depend upon the condition or disorder to be treated, the physiological effect desired, and the mode of administration. For example, the amount of antidepressant administered with the unconjugated estrogen compositions, depending on the antidepressant used, is about 0.75 to about 2 mg/24 hours, about 10 to about 20 mg/24 hours, or about 50 to about 100 mg/24 hours. Thus, in some aspects of the invention, the unconjugated estrogen compositions are administered with about 5 mg to about 120 mg/24 hours of fluoxetine hydrochloride. As another example, calcium administered with the unconjugated estrogen compositions can be in the form of calcium carbonate, at a daily dosage level of about 500 mg.

The unconjugated estrogen compositions of the present invention can also be produced in the form of a kit or package containing one or more dosage units. A dosage unit can be a single dosage form, e.g., a vaginal ring, or a transdermal patch. Alternatively, the dosage unit can be a container that contains multiple dosages of the unconjugated estrogen composition in the same dosage form, e.g., an ampoule containing multiple doses of the unconjugated estrogen composition in the form of a liquid for parenteral administration by injection, or a container containing multiple doses of the unconjugated estrogen composition in the form of a vaginal cream (e.g., a tube containing a month's supply of vaginal cream). The kit or package can contain one dosage unit, or multiple dosage units.

In some embodiments of the invention, the unconjugated estrogen compositions are provided as a pharmaceutical package containing multiple dosage units that are to be administered sequentially, in a consecutive manner, until the treatment period has ended or until the pack has been completed. If more than one package is to be used, the next package should be started on the next consecutive day. For example, the unconjugated estrogen compositions can be provided as a pharmaceutical package or kit containing three vaginal rings which are administered sequentially, each vaginal ring designed to deliver the unconjugated estrogens of the present invention continuously for a period of about 30 days, for a total treatment period of about 90 days. The first vaginal ring is administered beginning at day 1 of the treatment period and, after a period of 30 days, is subsequently replaced with the second vaginal ring in the pharmaceutical package (e.g., on day 31). After 30 days, the second vaginal ring is subsequently replaced with the third vaginal ring (e.g., on day 61). If the treatment period is to be extended, a second pharmaceutical package containing three vaginal rings can be started thereafter (e.g., on day 91).

The kits of the present invention can optionally contain a notice or printed instructions associated with the dosage units of the kits. For example, such printed instructions can be in a form prescribed by a governmental agency regulating the manufacture, use or sale of pharmaceuticals or biological products, which notice reflects approval by the agency of the manufacture, use or sale for human administration to treat a condition or disorder. Thus, in some embodiments, the kit further comprises printed matter, which, e.g., provides information on the use of an unconjugated estrogen composition of the present invention to treat a menopausal condition, or a pre-recorded media device which, e.g., provides information on the use of the unconjugated estrogen composition to treat a menopausal condition, or a planner.

“Printed matter” can be, for example, one of a book, booklet, brochure or leaflet. The printed matter can describe the use of the unconjugated estrogen compositions of the present invention for the treatment of a menopausal condition. Possible formats included, but are not limited to, a bullet point list, a list of frequently asked questions (FAQ) or a chart. Additionally, the information to be imparted can be illustrated in non-textual terms using pictures, graphics or other symbols.

“Pre-recorded media device” can be, for example, a visual media device, such as a videotape cassette, a DVD (digital video disk), filmstrip, 35 mm movie or any other visual media device. Alternately, pre-recorded media device can be an interactive software application, such as a CD-ROM (compact disk-read only memory) or floppy disk. Alternately, pre-recorded media device can be, for example, an audio media device, such as a record, audiocassette or audio compact disk. The information contained on the pre-recorded media device can describe the use of the unconjugated estrogen compositions of the present invention for the treatment of one or more of the conditions or disorders as described herein, e.g., a menopausal condition.

A “planner” can be, for example, a weekly, a monthly, a multi-monthly, a yearly, or a multi-yearly planner. The planner can be used as a diary to monitor dosage amounts, to keep track of dosages administered, or to prepare for future events wherein taking a regularly administered unconjugated estrogen composition of the present invention may be difficult. Alternately, the planner can be a calendar which will provide a means to monitor when a dosage has been taken and when it has not been taken. This type of planner will be particularly useful for patients having unusual schedules for administering medication to themselves. Additionally, the planner can be useful for the elderly, or other patient group who may administer medication to themselves and may become forgetful. One skilled in the art will appreciate the variety of planning tools that would be appropriate for use with the present invention.

The kit can also include a container for storing the other components of the kit. The container can be, for example, a bag, box, envelope or any other container that would be suitable for use in the present invention. Preferably, the container is large enough to accommodate each component and/or any administrative devices that may be necessary for a vaginal cream composition of the present invention. However, in some cases, it may be desirable to have a smaller container which can be hidden in a patient's pocketbook, briefcase or pocket.

The present invention is also directed to a method of delivering an unconjugated estrogen composition of the present invention to a patient in need thereof, the method comprising (a) registering in a computer readable storage medium the identity of a physician permitted to prescribe the unconjugated estrogen composition; (b) providing the patient with counseling information concerning the risks attendant to the unconjugated estrogen composition; (c) obtaining informed consent from the patient to receive the unconjugated estrogen composition despite the attendant risks; (d) registering the patient in a computer readable storage medium after obtaining their informed consent; and/or (e) permitting the patient access to the unconjugated estrogen composition.

Thus, for example, in some embodiments, the present invention is directed to a method of delivering an unconjugated estrogen composition of the present invention to a patient in need thereof, the method comprising:

(a) registering the patient in a computer readable storage medium; and

(b) permitting the patient access to the unconjugated estrogen composition.

The drug delivery methods of the present invention involve, inter alia, registering in a computer readable storage medium physicians who are qualified to prescribe the unconjugated estrogen compositions of the present invention. Once registered in the computer readable storage medium, the physician can be eligible to prescribe an unconjugated estrogen composition of the invention to a patient in need thereof. Generally speaking, in order to become registered in the computer readable storage medium, the physician may be required to comply with various aspects of, for example, providing patient education and counseling. The registration of the physician in the computer readable storage medium can be achieved by providing the physician, for example, by mail, facsimile transmission, or on-line transmission, with a registration card or form, preferably together with educational materials concerning the unconjugated estrogen compositions of the present invention. The physician can complete the registration card or form by providing information requested therein, and the registration card or form can be returned to the manufacturer or distributor of the unconjugated estrogen compositions, or other authorized recipient of the registration materials, for example, by mail, facsimile transmission or on-line transmission. The physician's information in the registration card or form is then entered into the computer readable storage medium. Suitable computer readable storage media which can be employed for registration of the physicians (as well as patients, as discussed below) will be apparent to one of ordinary skill in the art, once in possession of the teaching of the present application.

In the course of examination of a patient, including a patient suffering from a menopausal condition, the physician may determine that the patient's condition can be improved by the administration of an unconjugated estrogen composition of the present invention. Prior to prescribing the unconjugated estrogen composition, the physician can counsel the patient, for example, on the various risks and benefits associated with the unconjugated estrogen composition. The patient can be provided full disclosure of all the known and suspected risks associated with the unconjugated estrogen composition. Such counseling can be provided verbally, as well as in written form. In some embodiments, the physician can provide the patient with literature materials on the unconjugated estrogen composition, such as product information, educational materials, and the like.

In addition to receiving counseling on the risks attendant to unconjugated estrogen compositions of the present invention, the methods of the invention further require the patient to fill out an informed consent form which is signed by the patient. Upon the completion of the informed consent form, the patient can be registered in a computer readable storage medium. The computer readable storage medium in which the patient is registered can be the same as, or different from, the computer readable storage medium in which the physician is registered.

The registration into one or more computer readable storage media of the physician and patient, according to the methods describe herein, provides a means to monitor and authorize access to the unconjugated estrogen compositions of the present invention. Thus, the computer readable storage medium can serve to deny access to patients who fail to abide by the methods of the present invention. In some embodiments, access to an unconjugated estrogen composition of the invention is in the form of a prescription, wherein the prescribing physician is registered in a computer readable storage medium, has provided counseling to the patient concerning the attendant risks of the unconjugated estrogen composition, and has obtained informed consent from the patient, prior to prescribing the unconjugated estrogen composition to the patient in need thereof.

The present invention is also directed to methods of educating consumers about the use of an unconjugated estrogen composition of the invention, the method comprising distributing the unconjugated estrogen composition with consumer information at a point of sale. In some embodiments, the distribution will occur at a point of sale having a pharmacist or healthcare provider.

As used herein, the term “consumer information” can include, but is not limited to, an English language text, non-English language text, visual image, chart, telephone recording, audio recording, video recording, website, and access to a live costumer service representative. In some embodiments of the present invention, consumer information will provide directions for use of the unconjugated estrogen compositions of the present invention, appropriate age use, indication, contraindications, appropriate dosing, warnings, telephone number of website address. In some embodiments, the method further comprises providing professional information to relevant persons in a position to answer consumer questions regarding the unconjugated estrogen compositions.

As used herein, the term “professional information” includes, but is not limited to, information concerning the unconjugated estrogen compositions of the present invention designed to enable a healthcare professional to answer costumer questions regarding the vaginal cream composition.

A “relevant person,” as used herein, includes, for example, a physician, physician assistant, nurse practitioner, pharmacist and customer service representative.

All the various embodiments or options described herein can be combined in any and all variations. Other suitable modifications and adaptations of the variety of conditions and parameters normally encountered and obvious to those skilled in the art are within the spirit and scope of the invention. The following examples are illustrative, but not limiting, of the method and compositions of the present invention. Thus, the breadth and scope of the present invention should not be limited by any of the described exemplary embodiments, but should be defined only in accordance with the claims and their equivalents.

### EXAMPLE 1

The following general protocol can be used to prepare unconjugated estrogen gels of the invention for use as vaginal gels or for use in vaginal rings. Table II presents the ingredients for three representative unconjugated estrogen gels and the final weight percent range of each ingredient in each gel.

TABLE II
Unconjugated Estrogen Gels

| | Weight Percent (% (w/w)) | | |
|---|---|---|---|
| | of Component | | |
| Component | Sample Gel I | Sample Gel II | Sample Gel III |
| Part A | | | |
| Water | 10-80 | 10-80 | 10-80 |
| Carbomer 971P | 0.05-1.00 | 0.05-1.00 | 0.05-1.00 |
| Triethanolamine (TEA) | 0.1-5.00 | — | — |
| Part B | | | |
| Polyethylene Glycol 400 | 5-40 | 5-40 | 5-40 |
| (PEG) | | | |
| Propylene Glycol (PG) | 5-40 | 5-40 | 5-40 |
| Natrosol 250 HX | 0.5-10 | 0.5-10 | 0.5-10 |
| Part C | | | |
| Unconjugated estrogens | 0.1-15 | 0.1-15 | 0.1-15 |
| (Drug Mix) | | | |
| Ethanol | 1-30 | 1-30 | — |
| Glycerin | 5-40 | 5-40 | 5-40 |
| Preservative | 0-10 | 0-10 | 0-10 |
| Ethanol Rinse | As needed | — | — |
| Water Rinse | As needed | As needed | As needed |
| | qs to 100 | qs to 100 | qs to 100 |

Procedure:

Phase A is first prepared by dispersing Carbomer 971P in water in the amounts indicated in Table II with high agitation until the mixture is homogenous. This mixture is then heated to 45° C. with constant stirring. For the unconjugated estrogen gel of Example 1, TEA is added to the mixture once the viscosity of the mixture increases.

Phase B is next prepared by mixing together the indicated amounts of PEG 400 and PG until the mixture is uniform. Natrosol is then added to the mixture and mixed until dispersed. The resulting mixture is heated to 45° C.

Phase B is then added to Phase A and mixed until homogeneous. The resulting mixture is then removed the heat and stirred gently.

Phase C is prepared by mixing all the indicated ingredients in the amounts indicated in Table II. Phase C is then added to the combined Phase A/B mixture.

The beaker containing Phase C is then rinsed with rinse solution from those beakers originally containing Phase A and/or Phase B, and the rinsings are added to the combined Phase A/B/C mixture and mixed until incorporated to produce the final unconjugated estrogen gel.

### EXAMPLE 2

### Preparation of Unconjugated Estrogen Gel A

An unconjugated estrogen gel containing unconjugated equilin and unconjugated estrone in a 1:2 weight ratio, free of triethanolamine and ethanol, was prepared according to the protocol presented in Example 1 (sample gel II), as follows.

The following materials were used:

| Material | Source |
|---|---|
| Carbomer 971P | BF Goodrich |
| PEG-400 (PEG) | Sigma |
| Propylene Glycol (PG) | Spectrum |
| Natrosol 250 HX (HEC) | Hercules |
| Drug Mix | Previously Prepared |
| Ethanol (EtOH) | Fisher |
| Glycerin (Glycerin) | EM |
| DI (deionized) Water | Internal water system |
| Balance, toploader | Ohaus |
| Mechanical overhead mixer | Heidolph |
| Hotplate/stirrer | VWR |

Appropriate propeller/impellor/dissolver disk, weigh boats, various size beakers, droppers, spatulas

A 100× stock mixture of unconjugated equilin and unconjugated estrone in glycerin (“drug mix”) was first prepared by combining 1.56 g equilin with 3.13 g estrone and 95.31 g glycerin to form 100.00 mL of a 100× unconjugated estrogen stock mixture (0.0469 g (46.9 mg) unconjugated equilin and unconjugated estrone/g mixture, or a 4.69% stock mixture of unconjugated estrogens).

The components in Table IIA were then weighed out in the indicated amounts and combined with one another according to the protocol of Example 1 to produce unconjugated estrogen gel A.

TABLE IIA

| | Weight | | |
|---|---|---|---|
| | Percent | | |
| | (% (w/w)) | Amount | Amount |
| | of | Required | Weighed |
| Component | Component | (g/100 g) | (g) |
| Part A | | | |
| Water | 27.0 | 27.0 | 27.02 |
| Carbomer 971P | 0.50 | 0.50 | 0.50 |
| Part B | | | |
| PEG 400 | 20.0 | 20.0 | 20.03 |
| Propylene Glycol (PG) | 20.0 | 20.0 | 20.05 |
| Natrosol 250 HX | 1.50 | 1.50 | 1.51 |
| Part C | | | |
| Glycerin/Drug Mix | 1.00 | 1.00 | 1.00 |
| Ethanol | 10.0 | 10.0 | 10.03 |
| Glycerin | 10.0 | 10.0 | 10.00 |
| Water Rinse | 10.0 | 10.0 | 5.05 + 5.02 |
| Total | 100.00 | 100.00 | 100.20 |

The Part A components of Table IIA, 0.5 g Carbomer 971P and 27.02 g of water, were mixed together with high agitation until homogenous. The resulting mixture, “Phase A,” was heated to 45° C. with constant stirring.

The Phase B mixture was then prepared by mixing together 20.03 g of PEG 400 and 20.05 g PG (Part B components) until the mixture was uniform. 1.51 g of Natrosol 250 HX was then added and mixed until the Natrosol was dispersed. The resulting mixture, “Phase B,” was then heated to 45° C.

The Phase B mixture was then added to the Phase A mixture and mixed until homogeneous. The resulting “combined Phase A/B” mixture was then removed from the heat while it continued to be stirred gently.

The “Phase C” mixture was then prepared by mixing together the Part C components, 1.00 g of the previously prepared 1:2 unconjugated equilin/unconjugated estrone 100× stock mixture, 10.03 g of ethanol, and 10.00 g glycerin. The resulting “Phase C” mixture was then added to the combined Phase A/B mixture.

The beaker containing the Phase C mixture was then rinsed with a small amount of water, which was then combined with the water rinse from the beaker originally containing the Phase B mixture. The combined rinsings (total volume 10.07 g) were then mixed with the combined Phase A/B/C mixture to produce 100.20 g of the final unconjugated estrogen gel A.

### EXAMPLE 3

### Preparation of Unconjugated Estrogen Gel B

An unconjugated estrogen gel containing the unconjugated estrogens equilin and estrone in a 1:2 ratio (0.469 mg/g, or 0.0469% (w/w) total unconjugated estrogens), free of ethanol, was prepared according to the protocol presented in Example 1 (sample gel III), as follows.

The following materials were used:

| Material | Source |
|---|---|
| Carbomer 971P | BF Goodrich |
| PEG-400 (PEG) | Sigma |
| Propylene Glycol (PG) | Spectrum |
| Natrosol 250 HX (HEC) | Hercules |
| Drug Mix | Previously Prepared |
| Glycerin (Glycerin) | EM |
| DI Water | Internal water system |
| Balance, toploader | OHaus |
| Mechanical overhead mixer | Heidolph |
| Hotplate/stirrer | VWR |

Appropriate propeller/impellor/dissolver disk, weigh boats, various size beakers, droppers, spatulas

A 100× stock mixture of unconjugated equilin and unconjugated estrone in glycerin (“drug mix”) was first prepared as described in Example 2 above.

The components in Table IIB were then weighed out in the indicated amounts and combined with one another according to the protocol of Example 1 to produce unconjugated estrogen gel B, an example of sample gel III in Table II above.

TABLE IIB

| | Weight Percent | Amount | Amount |
|---|---|---|---|
| | (% (w/w)) of | Required | Weighed |
| Component | component | (g/100 g) | (g) |
| Part A | | | |
| Water | 30.0 | 30.0 | 30.07 |
| Carbomer 971P | 0.50 | 0.50 | 0.51 |
| Part B | | | |
| PEG 4000 | 20.0 | 20.0 | 20.03 |
| PG | 20.0 | 20.0 | 20.05 |
| Natrosol 250 HX | 2.0 | 2.0 | 2.07 |
| Part C | | | |
| Glycerin/Drug Mix | 1.00 | 1.00 | 1.01 |
| Glycerin | 16.5 | 16.5 | 16.52 |
| Water Rinse | 10.0 | 10.0 | 10.03 |
| Total | 100.00 | 100.00 | 100.29 |

The Part A components of Table IIB, 0.51 g Carbomer 971P and 30.07 g of water, were mixed together with high agitation until homogenous. The resulting Phase A mixture was heated to 45° C. with constant stirring.

The Phase B mixture was then prepared by mixing together 20.03 g of PEG 400 and 20.05 g PG (Part B components) until the resulting mixture was uniform. 2.07 g of Natrosol 250 HX was then added until the Natrosol was dispersed. The resulting Phase B mixture was then heated to 45° C.

The Phase B mixture was then added to the Phase A mixture until a homogeneous combined mixture resulted. The combined Phase A/B mixture was then removed from the heat while it continued to be stirred gently.

The Phase C mixture was then prepared by mixing together the Part C components, 1.01 g of the previously prepared 1:2 unconjugated equilin/unconjugated estrone 100× stock mixture and 16.52 g glycerin. The Phase C mixture was then added to the combined Phase A/B mixture.

The beaker containing the Phase C mixture was then rinsed with a small amount of water, which was then combined with the water rinse from the beaker originally containing the Phase B mixture. The combined rinsings (total volume 10.03 g) were then mixed with the combined Phase A/B/C mixture to produce 100.29 g of the final unconjugated estrogen gel B.

### EXAMPLE 4

The following protocols can be used to prepare unconjugated estrogen creams of the invention for use as vaginal creams or for use in vaginal rings. Table III lists the ingredients for each of five sample unconjugated estrogen creams, along with the final weight percent range of each ingredient in each cream.

TABLE III
Unconjugated Estrogen Creams

| | Weight Percent (% (w/w)) | | | | |
|---|---|---|---|---|---|
| | of Component | | | | |
| | Sample | Sample | Sample | Sample | Sample |
| Component | Cream I | Cream II | Cream III | Cream IV | Cream V |
| Phase A | | | | | |
| Distilled Water | 10-90 | 10-90 | 10-90 | 10-90 | 10-90 |
| Glycerin | 10-40 | 10-40 | 10-40 | 10-40 | 10-40 |
| Carbomer | — | 0.01-20 | — | 0.01-20 | — |
| Triethanolamine | — | 0.5-20 | — | 0.5-20 | — |
| (TEA) | | | | | |
| Sodium Lauryl | — | — | — | — | 0.05-10 |
| Sulfate | | | | | |
| Preservative | 0-10 | 0-10 | 0-10 | 0-10 | 0-10 |
| Sodium Phosphate | — | — | — | — | 0.01-10 |
| Dibasic | | | | | |
| Phase B | | | | | |
| Cetyl Palmitate | 1-40 | — | 1-40 | — | 1-30 |
| Cetyl Alcohol | 1-40 | 1-40 | 1-40 | 1-40 | 1-40 |
| Glyceryl | 0.01-20 | 0.01-20 | 0.01-20 | 0.01-20 | 0.01-20 |
| Monostearate | | | | | |
| Propylene Glycol | — | — | — | — | 0.5-50 |
| Monostearate | | | | | |
| Methyl Stearate | — | — | — | — | 0.5-30 |
| Promulgen D | 1-20 | 1-20 | 1-20 | 1-20 | — |
| Mineral Oil | — | — | — | — | 1-50 |
| Cottonseed Oil | 1-50 | — | — | — | — |
| Stearal | — | 1-20 | — | 1-20 | — |
| Solulan 16 | — | 0.1-10 | — | — | — |
| Solulan 98 | — | 0.1-10 | 0.1-10 | — | — |
| Lanolin | — | 1-40 | — | — | — |
| Petrolatum | — | — | 1-40 | — | — |
| White Wax | — | — | 0.5-50 | — | 0.5-50 |
| Suppocire AP | — | — | — | 1-40 | — |
| Gelucire 39/01 | — | — | — | 1-40 | — |
| 0.1 M Sodium | — | — | — | — | 0.1-20 |
| Hydroxide solution | | | | | |
| Unconjugated | 0.1-20 | 0.1-20 | 0.1-20 | 0.1-20 | 0.1-20 |
| Estrogens | | | | | |
| (Drug Mix) | | | | | |
| Water Rinse | As | As needed | As | As needed | As needed |
| | needed | qs to 100 | needed | qs to 100 | qs to 100 |
| | qs to 100 | | qs to 100 | | |

Sample Cream I

Phase A: Using high agitation, all Phase A ingredients and drug mix in the amounts indicated in Table III (sample cream I) are mixed together until uniform. The resulting Phase A mixture is heated to 80° C.

Phase B: All Phase B ingredients are combined and the resulting Phase B mixture is heated to 80° C. with gentle stirring.

The Phase B mixture is then added to the Phase A mixture and mixed with high agitation until one phase forms. The combined Phase A/Phase B mixture is then removed from the heat and the stirring speed decreased.

The drug mix container is then rinsed with a small amount of water, which is then added to the combined Phase A/Phase B mixture.

The resulting mixture is then cooled to 25-30° C. with continuous gentle stirring to form a cream.

Sample Cream II

Phase A: Using high agitation and shear, carbomer is added to water in the amounts indicated in Table III (sample cream II). Glycerin and the drug mix are then mixed together and added to carbomer solution until a uniform mixture results. The resulting Phase A mixture is then heated to 65-70° C.

Phase B: All Phase B ingredients in the amounts indicated in Table III (sample cream II) are combined together and then heated to 65-70° C. with gentle stirring to form the Phase B mixture.

The Phase B mixture is then added to the Phase A mixture and mixed with high agitation until one phase forms. The resulting combined Phase A/Phase B mixture is then removed from the heat and the stirring speed decreased. TEA is then added to the combined mixture as indicated in Table III.

The drug mix container is then rinsed with a small amount of water, and the rinse is added to the combined Phase A/Phase B mixture.

The mixture is then cooled to 25-30° C. with continuous gentle stirring to form sample cream II.

Sample Cream III

Phase A: Using high agitation, all Phase A ingredients and the drug mix are combined in the amounts indicated in Table III (sample cream III) and mixed together until uniform. The resulting Phase A mixture is then heated to 80° C.

Phase B: All Phase B ingredients are combined as indicated in Table III for forming the Phase B mixture, which is then heated to 80° with gentle stirring.

The Phase B mixture is then added to the Phase A mixture and mixed with high agitation until one phase forms. The resulting combined Phase A/Phase B mixture is then removed from the heat and the stirring speed decreased.

The drug mix container is then rinsed with a small amount of water and the rinsing is added to the combined Phase A/Phase B mixture.

The combined mixture is then cooled to 25-30° C. with continuous gentle stirring to form sample cream III.

Sample Cream IV

Phase A: Using high agitation and shear, the carbomer in dispersed in distilled water in the amounts indicated in Table III (sample cream IV). The indicated amounts of glycerin and drug mix are then combined, added to the carbomer mixture and then mixed until uniform. The resulting Phase A mixture is then heated to 65-70° C.

Phase B: All Phase B ingredients are combined in the amounts indicated in Table III to form the Phase B mixture, which is then heated to 65-70° C. with gentle stirring.

The Phase B mixture is then added to the Phase A mixture and mixed with high agitation until one phase forms. The resulting combined mixture is then removed from the heat and the stirring speed decreased. TEA is then added to the combined mixture.

The drug mix container is then rinsed with a small amount of water and the rinsings are added to the combined Phase A/Phase B mixture.

The resulting mixture is then cooled to 25-30° C. with continuous stirring to form sample cream IV.

Sample Cream V

Phase A: Water and glycerin are combined in the amounts indicated in Table III (sample cream V), and the remaining Phase A ingredients are then added. The resulting Phase A mixture is then heated to 70-80° C. with continuous mixing.

Phase B: All Phase B ingredients are combined in the amounts indicated in Table III and then heated to 70-80° C. with gentle stirring to form the Phase B mixture.

The Phase B mixture is then added to the Phase A mixture and mixed with high agitation until one phase forms. The temperature of the resulting combined Phase A/Phase B mixture is maintained at 70-80° C. Sodium hydroxide solution is added to the combined Phase A/Phase B mixture as indicated in Table III, and the resulting mixture is then removed from the heat and allowed to cool to 45° C.

At 45° C., the drug mix is then added to the combined Phase A/Phase B mixture. The drug mix container is then rinsed with a small amount of water and the rinsing is added to the combined mixture. The resulting mixture is then cooled to 25-30° C. and the pH is adjusted to 7.0-8.0 using sodium hydroxide solution to form sample cream V.

### EXAMPLE 5

### Preparation of Unconjugated Estrogen Cream A

An unconjugated estrogen cream (0.469 mg/g 1:2 unconjugated equilin:unconjugated estrone, or 0.0469% (w/w) total unconjugated estrogens) was prepared according to the protocol presented in Example 4, for sample cream I, as follows.

The following materials were used:

| Material | Source |
|---|---|
| Cetyl Palmitate | Spectrum |
| Cetyl Alcohol | Spectrum |
| Glyceryl Monostearate | Spectrum |
| Promulgen D | Amerchol |
| Cottonseed Oil | Sigma |
| Glycerin | EM |
| Drug Mix | Previously Prepared |
| Distilled Water | Internal Water System |
| Toploader Balance | OHaus |
| Mechanical overhead stirrer | Heidolph |
| Hotplate/stirrer (2) | VWR |

Appropriate propeller/impellor/dissolver disk, weigh boats, various size beakers, droppers, spatulas

A 100× stock mixture of unconjugated equilin and unconjugated estrone in glycerin (“drug mix”) was prepared as described in Example 2 above.

The components in Table IIIA in the indicated amounts were combined with one another according to the protocol of Example 4 to produce unconjugated estrogen cream A.

TABLE IIIA

| | Weight | | |
|---|---|---|---|
| | Percent | Amount | Amount |
| | (% (w/w)) of | Required | Weighed |
| Component | component | (g/100 g) | (g) |
| Phase A | | | |
| Distilled Water | 71.40 | 71.40 | 71.41 |
| Glycerin | 3.00 | 3.00 | 3.03 |
| Drug Mix | 1.00 | 1.00 | 1.00 |
| Phase B | | | |
| Cetyl Palmitate | 2.00 | 2.00 | 2.12 |
| Cetyl Alcohol | 3.00 | 3.00 | 3.12 |
| Glyceryl Monostearate | 2.60 | 2.60 | 2.72 |
| Promulgen D | 6.00 | 6.00 | 6.00 |
| Cottonseed Oil | 6.00 | 6.00 | 6.04 |
| Rinse Water | 5.00 | 5.00 | 5.00 |
| Total | 100.00 | 100.00 | 100.44 |

The Phase B ingredients in Table IIIA (2.12 g cetyl palmitate, 3.12 g cetyl alcohol, 2.72 g glyceryl monostearate, 6.00 g Promulgen D, and 6.04 g cottonseed oil) were combined and the resulting Phase B mixture was heated to 80° C. in a small beaker.

The Phase A component water (71.41 g) was placed in a stainless steel beaker, with an overhead mixer with disperser blade attached, and heated to 80° C.

Glycerin (3.03 g) and the drug mix (1.00 g of the 100× unconjugated equilin/unconjugated estrone glycerin stock mixture of Example 2) were combined in a small beaker then added to the water to form the Phase A mixture. The glycerin/drug beaker was then rinsed with 5.00 g water, which was added to the Phase A mixture.

The Phase B mixture was added to the Phase A mixture and stirred until uniform and until one phase was formed.

The combined Phase A/Phase B mixture was cooled to 25-30° C. using a water bath. The resulting unconjugated estrogen cream A was very smooth and off-white in color, with a light and fluffy texture.

### EXAMPLE 6

### Preparation of Unconjugated Estrogen Cream B

An unconjugated estrogen cream (0.469 mg/g 1:2 unconjugated equilin:unconjugated estrone, or 0.0469% (w/w) total unconjugated estrogens) was prepared according to the protocol presented in Example 4, for sample cream II, as follows.

The following materials were used:

| Material | Source |
|---|---|
| Carbomer 971P | BF Goodrich |
| Triethanolamine | JT Baker |
| Lanolin | Amerchol |
| Cetyl Alcohol | Spectrum |
| Stearal | Amerchol |
| Glyceryl Monostearate | Spectrum |
| Solulan 16 | Amerchol |
| Promulgen D | Amerchol |
| Solulan 98 | Amerchol |
| Glycerin | EM |
| Drug Mix | Previously Prepared |
| Distilled Water | Internal water system |
| Toploader balance | OHaus |
| Mechanical overhead stirrer | Heidolph |
| Hotplate/stirrer | VWR |

Disperser blade, spatulas, weigh boats, various size beakers, stainless steel beakers, droppers, stir bars

A 100× stock mixture of unconjugated equilin and unconjugated estrone in glycerin (“drug mix”) was prepared as described in Example 2 above.

The components in Table IIIB were combined with one another according to the protocol of Example 4 (for sample cream II in Table III) to produce unconjugated estrogen cream B.

TABLE IIIB

| | Weight | | |
|---|---|---|---|
| | Percent | Amount | Amount |
| | (% (w/w)) of | Required | Weighed |
| Component | Component | (g/100 g) | (g) |
| Phase A | | | |
| Distilled Water | 64.0 | 64.0 | 64.0 |
| Carbomer 971P | 0.15 | 0.15 | 0.15 |
| Glycerin | 2.00 | 2.00 | 2.00 |
| Drug Mix | 1.00 | 1.00 | 0.99 |
| Triethanolamine (TEA) | 0.85 | 0.85 | 0.85 |
| Phase B | | | |
| Lanolin | 10.00 | 10.00 | 10.00 |
| Cetyl Alcohol | 2.00 | 2.00 | 2.00 |
| Stearal | 1.50 | 1.50 | 1.50 |
| Glyceryl Monostearate | 1.00 | 1.00 | 1.01 |
| Solulan 16 | 3.00 | 3.00 | 3.00 |
| Promulgen D | 3.00 | 3.00 | 3.01 |
| Solulan 98 | 1.50 | 1.50 | 1.51 |
| Rinse Water (Drug Mix) | 5.00 | 5.00 | 5.01 |
| Rinse Water (TEA) | 5.00 | 5.00 | 4.99 |
| Total | 100.00 | 100.00 | 100.02 |

All Phase B ingredients (10.00 g lanolin, 2.00 g cetyl alcohol, 1.50 g stearal, 1.01 g glyceryl monostearate, 3.00 g Solulan 16, 3.01 g Promulgen D, and 1.51 g Solulan 98) were combined and heated to 65-70° C. in a small beaker. The resulting Phase B mixture was heated until all components were melted.

The Phase A distilled water (64.00 g) was placed in a stainless steel beaker and an overhead mixer with disperser blade was attached. Then 0.15 g of Carbomer 971P was dispersed in the water with high shear (400 rpm) and the resulting Phase A mixture was heated to 65-70° C.

Glycerin (2.00 g) and the drug mix (0.99 g of the 100× unconjugated equilin/unconjugated estrone glycerin stock mixture of Example 2) were mixed in a small beaker then added to the Phase A Carbomer/water mixture. The glycerin/drug beaker was rinsed with 5.01 g water, and the rinsing was added to the Phase A Carbomer/water mixture. Heating of the Phase A Carbomer/water mixture was then discontinued.

The Phase B mixture was then added to the Phase A Carbomer/water mixture, and the resulting combined mixture was stirred at 45° C. Then 0.85 g of TEA was added to the combined mixture, which was then stirred.

The combined mixture was cooled to 25-30° C. using a water bath. The resulting unconjugated estrogen cream B was very soft, with the consistency of a lotion.

### EXAMPLE 7

### Preparation of Unconjugated Estrogen Cream C

An unconjugated estrogen cream (0.469 mg/g 1:2 unconjugated equilin:unconjugated estrone, or 0.0469% (w/w) total unconjugated estrogens) was prepared according to the protocol presented in Example 4, for sample cream III, as follows.

The following materials were used:

| Material | Source |
|---|---|
| Cetyl Palmitate | Spectrum |
| Cetyl Alcohol | Spectrum |
| Glyceryl Monostearate | Spectrum |
| Promulgen D | Amerchol |
| Petrolatum | Fisher |
| Solulan 98 | Amerchol |
| White Wax | Spectrum |
| Glycerin | EM |
| Drug Mix | Previously Prepared |
| Distilled Water | Internal Water System |
| Toploader Balance | OHaus |
| Mechanical overhead stirrer | Heidolph |
| Hotplate/Stirrer (2) | VWR |

Disperser blade, spatulas, weight boats, various size beakers, stainless steel beakers, droppers, stir bars

A 100× stock mixture of unconjugated equilin and unconjugated estrone in glycerin (“drug mix”) was prepared as described in Example 2 above.

The components in Table IIIC were combined with one another according to the protocol of Example 4 (for sample cream III in Table III) to produce unconjugated estrogen cream C.

TABLE IIIC

| | Weight Percent | Amount | |
|---|---|---|---|
| | (% (w/w)) of | Required | Amount |
| Component | Component | (g/100 g) | Weighed (g) |
| Phase A | | | |
| Distilled Water | 61.80 | 61.80 | 61.80 |
| Glycerin | 3.00 | 3.00 | 3.01 |
| Drug Mix | 1.00 | 1.00 | 1.01 |
| Phase B | | | |
| Cetyl Palmitate | 3.00 | 3.00 | 3.00 |
| Cetyl Alcohol | 4.00 | 4.00 | 4.01 |
| Glyceral Monostearate | 3.50 | 3.50 | 3.50 |
| Promulgen D | 6.00 | 6.00 | 6.01 |
| Petrolatum | 3.00 | 3.00 | 2.99 |
| Solulan 98 | 0.70 | 0.70 | 0.72 |
| White Wax | 4.00 | 4.00 | 4.05 |
| Rinse Water | 10.00 | 10.00 | 10.00 |
| Total | 100.00 | 100.00 | 100.10 |

All Phase B ingredients (3.00 g cetyl palmitate, 4.01 g cetyl alcohol, 3.50 g glyceral monostearate, 6.01 g Promulgen D, 2.99 g petrolatum, 0.72 g Solulan 98, and 4.05 g white wax) were combined and heated to 80° C. in a small beaker.

The Phase A distilled water (61.80 g) was placed in a stainless steel beaker and an overhead mixer with dispenser blade was attached. The distilled water was heated to 80° C.

Glycerin (3.01 g) and drug mix (1.01 g of the 100× unconjugated equilin/unconjugated estrone glycerin stock mixture of Example 2) were mixed in a small beaker and then added to the Phase A distilled water. The glycerin/drug mix beaker was rinsed with 10.0 g water, and the rinsing was added to the Phase A water/glycerin/drug mixture.

The Phase B mixture was then added to the Phase A water/glycerin/drug mixture and stirred until uniform and until the resulting mixture formed one phase.

The combined mixture was cooled to 25-30° C. using a water bath to form unconjugated estrogen cream C. Cream C was a thick cream with a sturdy body and a smooth consistency and feel.

### EXAMPLE 8

### Preparation of Unconjugated Estrogen Cream D

An unconjugated estrogen cream (0.469 mg/g 1:2 unconjugated equilin:unconjugated estrone, or 0.0469% (w/w) total unconjugated estrogens) was prepared according to the protocol presented in Example 4, for sample cream IV, as follows.

The following materials were used:

| Material | Source |
|---|---|
| Carbomer 97 1P | BF Goodrich |
| Triethanolamine | JT Baker |
| Lanolin | Amerchol |
| Cetyl Alcohol | Spectrum |
| Stearal | Amerchol |
| Glyceryl Monostearate | Spectrum |
| Suppocire AP | Gattefosse |
| Promulgen D | Amerchol |
| Gelucire 39/01 | Gattefosse |
| Glycerin | EM |
| Drug Mix | Previously Prepared |
| Distilled Water | Internal Water System |
| Toploader Balance | OHaus |
| Mechanical overhead stirrer | Heidolph |
| Hotplate/Stirrer (2) | VWR |

Disperser blade, spatulas, weight boats, various size beakers, stainless steel beakers, droppers, stir bars

A 100× stock mixture of unconjugated equilin and unconjugated estrone in glycerin (“drug mix”) was prepared as described in Example 2 above.

The components in Table IIID were combined with one another according to the protocol of Example 4 (for sample cream IV in Table III) to produce unconjugated estrogen cream D.

TABLE IIID

| | Weight Percent | Amount | |
|---|---|---|---|
| | (% (w/w)) of | Required | Amount |
| Component | Component | (g/100 g) | Weighed (g) |
| Phase A | | | |
| Distilled Water | 49.00 | 49.00 | 49.12 |
| Carbomer 97 1 P | 0.50 | 0.50 | 0.53 |
| Glycerin | 2.00 | 2.00 | 2.13 |
| Drug Mix | 1.00 | 1.00 | 1.03 |
| Triethanolamine (TEA) | 1.50 | 1.50 | 1.52 |
| Phase B | | | |
| Lanolin | 6.00 | 6.00 | 6.00 |
| Cetyl Alcohol | 6.00 | 6.00 | 6.18 |
| Stearal | 2.00 | 2.00 | 1.97 |
| Glyceral Monostearate | 4.00 | 4.00 | 3.96 |
| Suppocire AP | 5.00 | 5.00 | 5.06 |
| Promulgen D | 3.00 | 3.00 | 3.14 |
| Gelucire 39/01 | 5.00 | 5.00 | 5.00 |
| Rinse Water (Drug Mix) | 5.00 | 5.00 | 5.15 |
| Rinse Water (TEA) | 5.00 | 5.00 | 5.03 |
| Rinse Water (Phase B) | 5.00 | 5.00 | 5.01 |
| Total | 100.00 | 100.00 | 100.83 |

All Phase B ingredients (6.00 g lanolin, 6.18 g cetyl alcohol, 1.97 g stearal, 3.96 g glyceral monostearate, 5.06 g Suppocire AP, 3.14 g Promulgen D, and 5.00 g Gelucire 39/01) were combined and heated to 65-70° C. in a small beaker. The mixture was heated until all ingredients were melted.

The Phase A distilled water (49.12 g) was placed in a stainless steel beaker and an overhead mixer with dispenser blade was attached. 0.53 g Carbomer 971P was dispersed in the distilled water with high shear (400 rpm) and the carbomer/water Phase A mixture was heated to 65-70° C.

Glycerin (2.13 g) and drug mix (1.03 g of the 100× unconjugated equilin/unconjugated estrone glycerin stock mixture of Example 2) were mixed together in a small beaker then added to the carbomer/water Phase A mixture. The glycerin/drug beaker was rinsed with 5.15 g of water and the rinse water was added to the carbomer/water/drug mix Phase A mixture. Heating of the Phase A mixture was discontinued.

The Phase B mixture was added to the Phase A mixture, and the resulting combined mixture was stirred. When the mixture reached 45° C., TEA was added and the mixture stirred. The combined mixture was then cooled to 25-30° C. using a water bath to form unconjugated estrogen cream D. Cream D was very smooth, with a sturdy body and good viscosity and feel.

### EXAMPLE 9

Vaginal rings with cores containing the unconjugated estrogen compositions of the invention can be prepared using the following general protocol. The protocol describes the preparation of vaginal rings using a sequential co-injection method disclosed in U.S. Pat. No. 6,394,094 (McKenna et al.). Table IV presents the weight percentages of the components of each vaginal ring.

TABLE IV
Unconjugated Estrogen Co-injected Vaginal Rings

| Component | Weight Percent (% (w/w)) |
|---|---|
| Unconjugated estrogens (Drug Mix) | 0.01-60 |
| Silicone Elastomer | 40-99.99 |

Silicone polymer vaginal rings containing 0.01-60% (w/w) unconjugated estrogens can be prepared as follows:

A core mix (comprising between 0.01% (w/w) to 60% (w/w) of drug mix) is first prepared by compounding the desired amounts of unconjugated estrogens with appropriate amounts of a two-part silicone elastomer. The shell material is prepared next from plain silicone elastomer, free of unconjugated estrogens.

A short shot of the shell material is then injected into a heated, compressed toroidal mold. Shortly afterward, the core material is injected into the mold, pushing the shell material further into the mold until a ring is formed. The shot size of the core material can be varied to change the geometry and/or the dosage of the resultant ring.

The ring is allowed to cure under heat for a specified amount of time before it is removed from the mold. The process is repeated for subsequent rings.

One skilled in the art would understand that, despite the full description provided herein, the present invention can be performed within a wide and equivalent range of conditions, formulations, and other parameters without affecting the scope of the invention or any embodiment thereof. All patents, patent applications, and publications cited herein are fully incorporated by reference herein in their entirety.

What is claimed is:

1. An unconjugated estrogen composition comprising two or more unconjugated estrogens wherein at least one unconjugated estrogen is selected from the group consisting of unconjugated equilin, unconjugated 17α-dihydroequilin, unconjugated 17β-dihydroequilin, and combinations thereof, and wherein the unconjugated estrogen composition is substantially free of conjugated estrogens.

2. The unconjugated estrogen composition of claim 1, wherein the composition comprises unconjugated equilin.

3. The unconjugated estrogen composition of claim 1, further comprising an unconjugated estrogen selected from the group consisting of unconjugated 17α-estradiol, unconjugated Δ^{8,9}-dehydroestrone, unconjugated 17β-estradiol, unconjugated estrone, unconjugated equilenin, unconjugated 17α-dihydroequilenin, unconjugated 17β-dihydroequilenin and combinations thereof.

4. The unconjugated estrogen composition of claim 3, wherein the composition comprises unconjugated equilin and unconjugated estrone.

5. The unconjugated estrogen composition of claim 3, wherein the composition comprises unconjugated equilin, unconjugated 17α-dihydroequilin, unconjugated 17β-dihydroequilin, unconjugated 17α-estradiol, unconjugated equilenin, unconjugated 17α-dihydroequilenin, unconjugated 17β-dihydroequilenin, unconjugated 17β-estradiol and unconjugated estrone.

6. The unconjugated estrogen composition of claim 1, wherein the composition is in an anal suppository, buccal, parenteral, transdermal, vaginal, nasal, topical, implantable, or subcutaneous dosage form.

7. The unconjugated estrogen composition of claim 6, wherein the dosage form has a rate of release of about 0.01 μg/day to about 2000 μg/day of the unconjugated estrogen.

8. The unconjugated estrogen composition of claim 6, wherein the composition is in a vaginal dosage form.

9. The unconjugated estrogen composition of claim 8, wherein the vaginal dosage form is a vaginal ring.

10. The unconjugated estrogen composition of claim 9, wherein the vaginal ring has a rate of release of about 5 μg/day to about 200 μg/day of the unconjugated estrogen.

11. The unconjugated estrogen composition of claim 9, wherein the vaginal ring comprises a polymer core containing the unconjugated estrogen composition and a polymer sheath that surrounds the polymer core.

12. A method of treating a peri-menopausal or menopausal condition in a female in need thereof, the method comprising administering the composition of claim 9, wherein the vaginal ring releases the unconjugated estrogen at a rate of about 5 μg/day to about 200 μg/day, and wherein the vaginal ring is administered to the female for a period of about 30 days to about 90 days.

13. The method of claim 12, wherein the peri-menopausal or menopausal condition is selected from the group consisting of vaginal dryness, vaginal atrophy, vulvar atrophy, atrophic vaginitis, vaginal pruritus, dyspareunia, and dysuria.

14. The method of claim 13, wherein the peri-menopausal or menopausal condition is vaginal atrophy.

15. A method of treating a condition resulting from hypoestrogenism in a female in need thereof, the method comprising administering to the female a therapeutically effective amount of the unconjugated estrogen composition of claim 1.

16. The method of claim 15, wherein the condition resulting from hypoestrogenism is a peri-menopausal or menopausal condition.

17. The method of claim 16, wherein the peri-menopausal or menopausal condition is selected from the group consisting of vaginal dryness, vaginal atrophy, vulvar atrophy, atrophic vaginitis, vaginal pruritus, dyspareunia, and dysuria.

18. The method of claim 17, wherein the peri-menopausal or menopausal condition is vaginal atrophy.

19. A method of delivering the unconjugated estrogen composition of claim 1 to a patient in need thereof, the method comprising:
(a) registering the patient in a computer readable storage medium, and
(b) permitting the patient access to the unconjugated estrogen composition.

20. The method of claim 19, wherein the access to the unconjugated estrogen composition is through a prescription.
